(19) United States
(12) Patent Application Publication
(10) Pub. No.: US 2008/0160514 A1
(43) Pub. Date: Jul. 3, 2008

(54) Enzymatic treatment of pulp

(75) Inventors: James E. Sealey, Perry, GA (US); Jennifer C. Luk, Centerville, GA (US); Roger O. Campbell, Auburn, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: Weyerhaeuser Co., Federal Way, WA (US)

(21) Appl. No.: 11/616,719

(22) Filed: Dec. 27, 2006

(51) Int. Cl. C12N 7/06

(52) U.S. Cl. 435/6

(57) ABSTRACT

Carbon dioxide is used to adjust the pH of cellulosic pulp or biomass to 6.5 to 7.5 prior to or during the application of enzymes to and the interaction of the enzymes with the cellulosic pulp. The carbon dioxide is applied as a gas into a pulp stream or biomass and tends to disperse more uniformly throughout the pulp or biomass.

The Field of the invention is the enzymatic treatment of cellulosic pulp.

Enzymes are used in the treatment of cellulosic pulp to improve the bleaching of pulp. One such treatment is in conjunction with the use of chlorine dioxide for cellulosic pulp bleaching. The use of enzymes will give greater efficiency to the use of the chlorine dioxide. In one embodiment less chlorine dioxide may be used if the cellulosic pulp is treated with an enzyme, reducing the cost of bleaching. In another embodiment a greater brightness may be achieved with the same amount of chlorine dioxide if the cellulosic pulp is treated with an enzyme.

Another use of enzymes is to control the viscosity of the pulp during the bleach treatment. A low uniform viscosity is needed for dissolving pulps useful for rayon or lyocell production. Enzymes may be used to control this viscosity.

Another use of enzymes is to increase the drainage of the pulp during dewatering and drying. A pulp that has better draining characteristics can be dewatered more quickly.

Another use of enzymes is to improve ethanol conversion rates when converting biomass to ethanol.

Enzymes that are useful with cellulose are xylanases, cellulases, hemicellulases, peroxidases, mannases, laccases (oxidoreductases), lipases and combinations of these enzymes.

The cellulose pulp or any biomass must be at the correct pH in order for the enzymes to work. The usual pH is 6.5 to 7.5. An acid, such as sulfuric, nitric or hydrochloric acid, is usually used to adjust to the appropriate pH. There are problems associated with the use of these mineral acids. The mineral acids tend to harden the outside of and reduce the void volume within cellulose pulp fibers and make it more difficult to for the enzymes to interact with the cellulose pulp fibers. Mineral acids are applied as a liquid and the dispersion of the acid through the pulp or biomass can be non-uniform.

The inventors have discovered that the use of carbon dioxide to adjust the pH of the cellulose pulp fiber or biomass to the correct pH of 6.5 to 7.5 does not create the problems that the use of mineral acids do. The carbon dioxide tends to maintain the openness of the cellulose pulp fiber or biomass and allow better interaction of the enzyme with both the outside and the inside of the cellulose pulp fiber or biomass. The carbon dioxide is applied as a gas and tends to disperse more uniformly through the fiber or biomass.

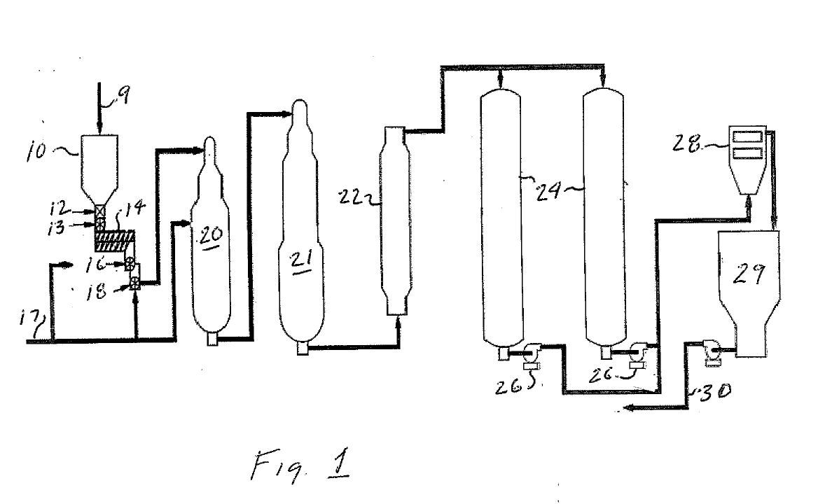

FIG. 1 is a diagram of a pulp mill digester and brown stock washing system.

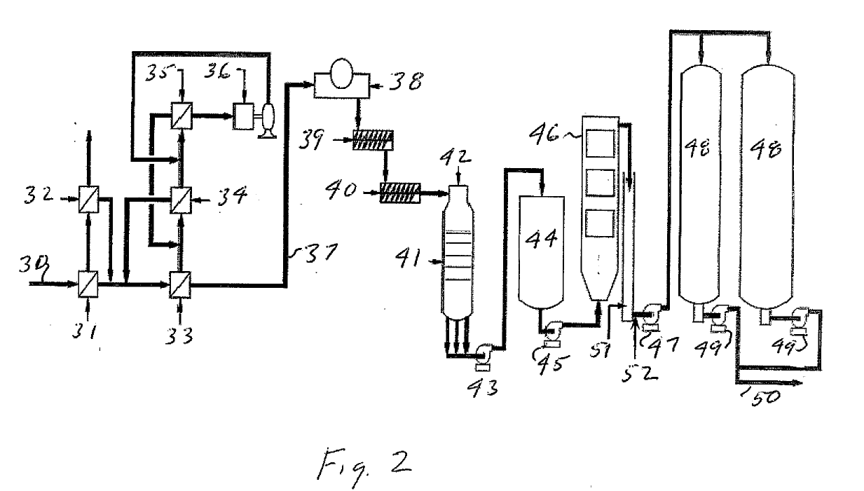

FIG. 2 is a diagram of a pulp mill pre-bleach system.

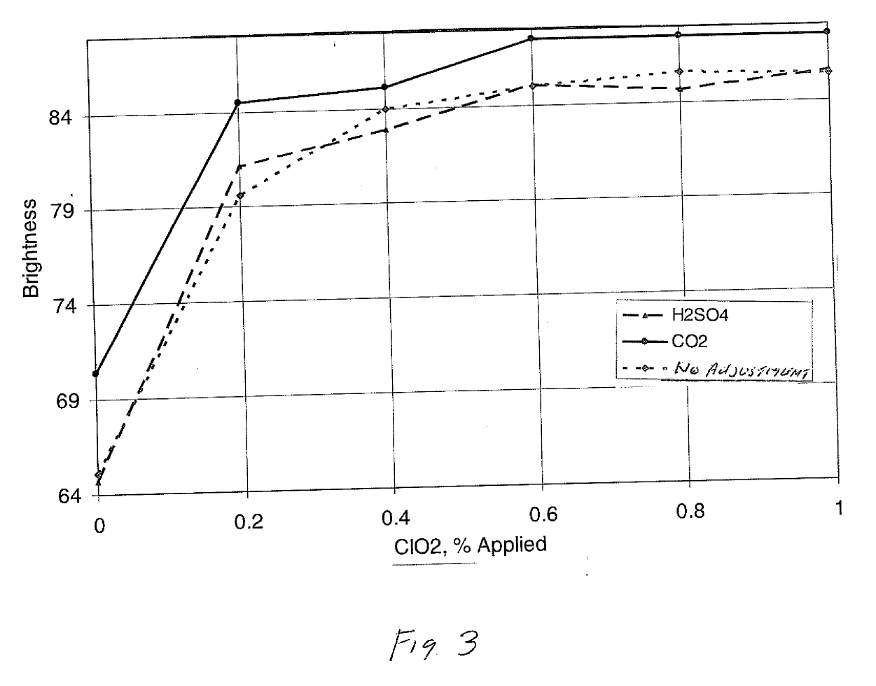

FIG. 3 is diagram of brightness vs. chlorine dioxide use.

Carbon dioxide is used to adjust the pH of cellulosic pulp or biomass to 6.5 to 7.5 prior to or during the application of enzymes to and the interaction of the enzymes with the cellulosic pulp. The carbon dioxide is applied as a gas into a pulp stream or biomass and tends to disperse more uniformly throughout the pulp or biomass.

Enzymes that are useful with cellulose are xylanases, cellulases, hemicellulases, peroxidases, mannases, laccases (oxidoreductases), lipases and combinations of these enzymes. Xylanases may be sold under trademarks such as the Diversa xylanase sold under the trademark Luminase™ and the AB Enzymes xylanase sold under the trademark Ecopulp®.

The lignocellulosic materials that may be use with the enzyme is selected from softwood materials and hardwood materials. The softwood and hardwood material may include spruce, fir, pine, hemlock, ash, birch, beech, oak, alder, maple, aspen, and poplar. Other softwood and hardwoods may be used, depending on the use and location. Non-wood lignocellulosic materials, for example, kenaf, jute, hemp, flax, ramie bagasse and rice straw, may be used as a lignocellulosic material for the method of the present invention.

The enzymes have a number of uses in the treatment of cellulosic pulp.

Enzymes are used in the treatment of cellulosic pulp to improve the bleaching of pulp. One such treatment is the use of chlorine dioxide for cellulosic pulp bleaching. In one embodiment the use of an enzyme will give greater efficiency to the use of the chlorine dioxide. Less chlorine dioxide may be used if the cellulosic pulp is treated with an enzyme and this can reduce the cost of bleaching and reduce environmental impacts. In another embodiment the use of an enzyme will allow a greater brightness to be achieved with the same amount of chlorine dioxide.

Another use of enzymes is to control the viscosity of the pulp during the bleach treatment. A low uniform viscosity is needed for dissolving pulps useful for rayon or lyocell production. Enzymes may be used to control this viscosity by reducing the length of the cellulose chain.

Another use of enzymes is to increase the drainage of the pulp during dewatering and drying. A pulp that has better draining characteristics can be dewatered more quickly

The pulp usable for the present invention may be a chemically cooked pulp, namely a chemical pulp. Either a sulfate or sulfite chemical process may be used. Mechanical or thermomechanical pulp is also usable for the method of the present invention. Also, the pulp usable for the present invention includes used recycled paper pulp made from the above-mentioned pulps.

The pulp is treated with carbon dioxide to adjust the pH to a level of 6.5 to 7.5 and the pulp is thereafter treated with an enzyme. The enzyme may be used to increase the brightness of the pulp, to increase the efficiency of the chlorine dioxide use, to adjust the viscosity of the pulp or increase the drainage of the pulp.

In the laboratory experiments, the use of carbon dioxide was compared against sulfuric acid or other mineral acids. In this case a high pH pulp was adjusted with sulfuric acid. This was compared with a pulp treated with carbon dioxide. The brightness (TAPPI standard test T452) was used to determine the pulp response to the enzyme treatment. The brightness correlates to the remaining lignin and is a predictor of the amount of bleaching chemical needed for target brightness. The brightness test was used because it is an easily measured property correlating to residual lignin. The higher enzyme activity in the carbon dioxide treated pulps was shown by the higher brightness as shown in FIG. 3.

### EXAMPLE 1

Southern pine kraft pulp pulped and treated with oxygen as shown in FIG. 1 was treated with CO₂ by injecting the CO₂ into a pulp stream at the mill as shown in FIG. 2 to provide a pulp having a pH of 7.1. A sample of the pulp was treated with the equivalent of 1 pound of Luminase™ per ton of pulp. The sample were treated with varying levels of chlorine dioxide for 240 minutes at 70° C. The results of these treatments are shown in FIG. 3.

### EXAMPLE 2

Southern pine kraft pulp pulped and treated with oxygen as shown in FIG. 1 was treated with sulfuric acid by adding sulfuric acid to the pulp to provide a pulp having a pH of 7.1. A sample of the pulp was treated with the equivalent of 1 pound of Luminase™ per ton of pulp. The samples were treated with varying levels of chlorine dioxide for 240 minutes at 70° C. The results of these treatments are shown in FIG. 3.

### EXAMPLE 3

A sample of southern pine kraft pulp pulped and treated with oxygen as shown in FIG. 1 but not adjusted to a pH of 7.1 was also provided. A sample of the pulp was treated with the equivalent of 1 pound of Luminase™ per ton of pulp. The samples were treated with varying levels of chlorine dioxide for 240 minutes at 70° C. The results of these treatments are shown in FIG. 3.

It can be seen that brightness of the carbon dioxide treated pulp in Example 1 was higher before bleaching and remained higher during bleaching than either the sulfuric acid treated pulp of example 2 or the untreated pulp of example 3.

FIGS. 1 and 2 is a diagram of part of a typical pulp mill digestion system and a pulp mill pre-bleach system. The bleach system and the recovery system of a pulp mill are not shown. There are many forms of pulp mill systems and the one shown in FIGS. 1 and 2 is illustrative only.

Wood chips are placed in a chip bin 10, metered by a chip meter 12 and fed by a low pressure feeder 13 into a steaming vessel 14 where the chips are steamed. An auger steaming vessel is shown. Rocks and other debris are separated from the steamed chips in the rock separator 16.

White liquor 17, a solution of sodium hydroxide and sodium sulfide in an appropriate ratio for pulping, is added to the chips and the chips fed by a high pressure feeder 18 into an impregnation vessel 20. Additional white liquor is added to the chips in the impregnation vessel 20. The chips are then transferred to a digester 21 in which the chips are cooked under pressure and the wood reacted with the sodium hydroxide and sodium sulfide.

The cooked chips are discharged from the digester 21. The pressure on the chips is released during discharge and this fiberizes the chips. The fiberized chips pass to a washer 22 and the pulp fibers are separated from the black liquor, the cooking solution which also contains lignin and hemicellulose. The pulp fibers then pass to surge tanks 24, and are pumped by pumps 26 into a brownstock washer 28. The washer 28 removes more of the cooking liquor, lignin and hemicellulose from the pulp fibers. The pulp fibers are then transported to a high density storage tank 29.

The pulp fibers 30 from the tank 29 then pass through primary and secondary knotters 31 and 32 which fiberize knots, bunches of fibers that were not previously fiberized. The pulp fibers then pass through primary, secondary and tertiary screens 33, 34, and 35 which separate the knots from the fibers. The remaining knots pass through a refiner 36 which fiberizes the remaining knots. The purpose of this portion of the system is to turn the knots into fibers and only have fibers passing through the rest of the system.

The pulp fibers 37 are then dewatered in a decker 38 and pass through a steam mixer 39 and a screw feeder 40 into an oxygen reactor 41 in which the pulp fibers are further delignified. The fibers are fluffed by a fluffer 42 before entering the oxygen reactor 41.

The pulp fibers are discharged from the oxygen reactor 41 and pumped by a medium consistency pump 43 to a blow tank 44. From the blow tank 44 the fibers are pump by a medium consistency pump 45 upward through a post oxygen diffuser washer 46 in which the lignin and hemicellulose is further washed from the cellulose fibers. The cellulose pulp fibers are then pumped by medium consistency pumps 47 to pre-bleach surge tanks 48. The fibers 50 are then pumped by pumps 49 to the bleach stage.

The bleach stage may consist of any combination of bleach units. Bleach units are typically oxidative stages followed by extraction and washing stages. The oxidative stages are typically chlorine (C), chlorine dioxide (D) and hypochlorite (H). The extraction and washing stages usually use sodium hydroxide to extract the lignin and other material followed by water washing (E_{OP}). Typical bleaching sequences are C-E_{OP}-D, D-E_{OP}-D, D-E_{OP}-D-E_{OP}-D, and D-E_{OP}-D-H. The sequences may also include ozone (Z) and peroxide (P) stages.

The enzyme may be added to the vacuum side of pumps 26 or 47. The carbon dioxide would be added at the same time. The enzyme 51 and the carbon dioxide 52 are shown being added before the pump 47. There is a pressure head after each of these pumps that will allow good dispersion of the enzyme through and into the cellulose pulp. The temperature of the pulp slurry should not be so high as to cause the enzyme to lose its activity or denature. A temperature around 160° F. (71° C.) will maintain the viability of the enzyme. The dosage of enzyme will depend on the enzyme being used and the purpose for its use.

There is a control loop in the brownstock washer 28 and storage tank 30 or the post oxygen diffusion washer 46 and the surge tanks 48 which senses the pH of the pulp slurry and adds carbon dioxide to maintain the pH between 6.5 and 7.5.

The system was tested in a mill environment to determine whether there was chlorine dioxide efficiency. There were two one-week trials. The second one-week trial was the better of the two because there was better understanding of the process. The following data is from the second week trial. It was discovered that there was both chlorine dioxide and sodium hydroxide efficiency.

The pulp mill had a DE_{OP}D bleach stage and the enzyme and carbon dioxide were added as shown in FIG. 2. The pulp passed through the washer 46 into the surge tank 48 where it has at least 4 hours retention time. The pH in the washer 46 was controlled to approximately 7 and the temperature was controlled to approximately 160° F. The average temperature in the standpipe to the post oxygen washer 46 was 162.1° F. The enzyme was Luminase™. It was added at a dosage rate of 131 g/air dry metric ton of pulp. This is an estimate because there was no device to measure the rate of flow of pulp into the surge tank. The flow into the surge tank was estimated by measuring the flow of pulp slurry into the bleach plant, the changes of level in the surge tank, the exit flow of the surge tank and the consistency of the pulp slurry (the weight of pulp in the pulp/water slurry). This estimate was used to determine the ml/min of enzyme to add to the pulp slurry at the medium density pump. The amount of chlorine dioxide to the first chlorine dioxide tower was reduced 17.1% and the amount of chlorine dioxide to the second chlorine dioxide tower was reduced 6.6%. The amount of sodium hydroxide (caustic) added to the E stage was reduced 22.5%.

Enzymes are also used to improve the conversion of biomass to ethanol. Carbon dioxide may also be used to adjust the pH of the biomass in ethanol conversion and decrease the enzyme doses and or time for conversion of cellulose to AHG (glucose).

While embodiments of the invention has been illustrated and described, it will be appreciated that various changes can be made therein without departing from the spirit and scope of the invention.

The embodiments of the invention in which an exclusive property or privilege is claimed are defined as follows:

1. A method for treating a cellulosic pulp in a slurry comprising:
treating the cellulosic pulp in a slurry with an enzyme, and
adjusting the pH of the cellulosic pulp in a slurry for the enzyme treatment by the addition of carbon dioxide.

2. The method of claim 1 wherein the enzyme is selected from xylanases, cellulases, hemicellulases, peroxidases, mannases, laccases, lipases and combinations of these enzymes.

3. The method of claim 1 wherein the pH is adjusted to a pH of 6.5 to 7.5.

4. The method of claim 3 wherein the enzyme is selected from xylanases, cellulases, hemicellulases, peroxidases, mannases, laccases, lipases and combinations of these enzymes.

5. The method of claim 1 further comprising
bleaching the cellulosic pulp after treating the cellulosic pulp with the enzyme.

6. The method of claim 5 wherein the enzyme is selected from xylanases, cellulases, hemicellulases, peroxidases, mannases, laccases, lipases and combinations of these enzymes.

7. The method of claim 5 wherein the bleaching of the cellulosic pulp uses chlorine dioxide as the bleaching chemical.

8. The method of claim 7 wherein the enzyme is selected from xylanases, cellulases, hemicellulases, peroxidases, mannases, laccases, lipases and combinations of these enzymes.

9. The method of claim 1 wherein the viscosity of the pulp is adjusted through the use of the enzyme.

10. The method of claim 9 wherein the enzyme is selected from xylanases, cellulases, hemicellulases, peroxidases, mannases, laccases, lipases and combinations of these enzymes.

11. A method for treating a biomass comprising:
treating the biomass with an enzyme, and
adjusting the pH of the biomass for the enzyme treatment by the addition of carbon dioxide.

12. The method of claim 11 wherein the enzyme is selected from xylanases, cellulases, hemicellulases, peroxidases, mannases, laccases, lipases and combinations of these enzymes.

13. The method of claim 11 wherein the biomass is converted to ethanol.

14. The method of claim 13 wherein the enzyme is selected from xylanases, cellulases, hemicellulases, peroxidases, mannases, laccases, lipases and combinations of these enzymes.
